(19) United States
(12) Patent Application Publication
(10) Pub. No.: US 2019/0021695 A1
(43) Pub. Date: Jan. 24, 2019

(54) HOLDING DEVICE FOR AN IMAGING TRANSDUCER AND A SYSTEM FOR MEASURING FLOW MEDIATED DILATION

(71) Applicant: Indiana State University, Terre Haute, IN (US)

(72) Inventors: Amy K. Watts, Robinson, IL (US); Francisco J. Morales, Terre Haute, IN (US); Alvaro N. Gurovich, Terre Haule, IN (US); Joseph O. West, Terre Haute, IN (US)

(73) Assignee: Indiana State University, Terre Haute, IN (US)

(21) Appl. No.: 16/041,522

(22) Filed: Jul. 20, 2018

Related U.S. Application Data
(60) Provisional application No. 62/535,708, filed on Jul. 21, 2017.

(51) Int. Cl. A61B 8/00; A61B 8/14; A61B 8/06

(52) U.S. Cl. CPC A61B 8/4227; A61B 8/06; A61B 8/14

(57) ABSTRACT

Disclosed is an imaging transducer holding device for use with a patient that includes a holding structure which holds an imaging transducer and an attachment portion which attaches the holding structure to a patient. The holding device is useful for assessing the condition of a patient's blood vessel via ultrasound imaging. Also disclosed are a system and a method for imaging the vascular condition of a patient, the system containing the holding device and an imaging transducer.

### CROSS-REFERENCE TO A RELATED APPLICATION

The present application claims the benefit of U.S. Provisional Patent Application No. 62/535,708, filed Jul. 21, 2017, the disclosure of which is incorporated herein in its entirety for all purposes.

### BACKGROUND OF THE INVENTION

Cardiovascular diseases (CVD), including coronary artery disease and stroke, are the leading cause of death in the Western World, with a direct and indirect cost of more than $430 billion in the United States. The first step of the development of these diseases is a process known as endothelial dysfunction (ED) in which the inner layer of the arteries are unable to generate several biochemical protective molecules necessary to maintain homeostasis. Flow mediated dilation (FMD) is a clinical, noninvasive way to test for ED.

Atherosclerosis, the pathological deterioration of endothelium that results in vessel blockage, is responsible for approximately 90% of all cardiovascular diseases [1, 2]. Endothelial dysfunction is the first step in this process, defined by the decrease in nitric oxide availability within the endothelium, developing oxidative stress. When endothelial oxidative stress is increased, endothelial NO synthase is uncoupled, and generates a decrease in NO production and bioavailability [3]. FMD is a non-invasive assessment of endothelial function and vasodilatory capacity in humans [4-6] with an immense potential to become a clinical tool for cardiovascular risk assessment [1]. Therefore, FMD is normally considered as a biomarker for endothelial function and NO bioavailability in humans, relevant to study cardiovascular risk factors such as hypertension [5, 7]. There are three major sites where FMD is assessed. The most studied one is the brachial artery [4, 5]; however, femoral artery [8] and popliteal artery [9] sites have been used to study large elastic and leg conduit arteries, respectively.

FMD uses ultrasound imaging of the arm's brachial artery to test artery's dilatory capacity. Recent publications from the Cardiovascular Health Study confirmed FMD as strong clinical predictor for cardiovascular events. Although FMD has been used in research for more than 20 years showing significant results relevant to vascular physiology, it has not been able to translate its performance to the doctor's office. The main problem in this translation is a lack of standardization and a prolonged learning curve. The problem is not solved due to the lack of a device that is convenient to use on a patient and that provides reliable vascular assessment during ultrasound diagnostics.

Vascular assessments using ultrasound imaging, such as FMD, are operator-dependent because a trained sonographist must hold the probe of the ultrasound in the same position continuously for about 10 minutes on the same place. This task is very difficult to accomplish because the hand of the operator will fatigue after a couple of minutes and will affect the measurements. Also, there is some risk of developing tendinopathies on the sonographer if the procedure is repeated too many times during a given period.

An external stereotaxic arm has been used to help collecting FMD but it has two main inconveniences: first, if the patients move their arm (as it is often difficult to stay still for 5 minutes or more), the image will be lost, and second, the sonographist still must maintain his or her hand on the probe all the time to ensure a good quality of the image, which will be cumbersome.

The foregoing shows that there exists an unmet need for a system and method for reliably measuring the flow mediated dilation of a blood vessel in a patient.

There further exists an unmet need for a transducer holding device that is convenient to use on a patient during ultrasound diagnostics and that provides reliable vascular assessment of ED.

### BRIEF SUMMARY OF THE INVENTION

The present invention provides an imaging transducer holding device for use with a patient comprising a holding structure which holds an imaging transducer and an attachment portion which attaches the holding structure to a patient.

The present invention further provides a system for determining the condition of a blood vessel, e.g., the flow mediated dilation of a blood vessel, in a patient comprising a device as described above and an ultrasonic probe.

The present invention further provides a method of diagnosing a condition of a blood vessel, e.g., the flow mediated dilation of a blood vessel, in a patient comprising securing a device as described above clamps on the upper arm of the patient, securing an ultrasonic probe into the device, and measuring the condition of the blood vessel, e.g., flow mediated dilation of the blood vessel.

The holding device of the present invention assists with vascular assessment via ultrasound imaging. The holding device allows more practitioners to use FMD testing for earlier detection of cardiovascular development by decreasing the variability of vascular imaging. Little or no external human manipulation is needed during the imaging procedure once the device is properly positioned. The holding device improves the accuracy and reproducibility of non-invasive clinical tests generally, such as FMD through ultrasound imaging to detect cardiovascular diseases.

The holding device of the invention can be built according to the specifications of the US transducer industry. The holding device supports the detection head of ultrasound mechanisms, and allows for a hands-free application of the device with x, y, z, and angular adjustments for location and pressure on the subject.

### BRIEF DESCRIPTION OF THE SEVERAL VIEWS OF THE DRAWINGS

FIG. 1 is a perspective view of the holding device and transducer attached to the arm of a patient.

FIG. 2 is an exploded view of first embodiment of the holding device.

FIG. 3 is an exploded view of a second embodiment of the holding device.

FIG. 4 is an exploded view of a third embodiment of the holding device.

FIG. 5 depicts a screenshot of a sonogram of the artery present in a patient's upper arm generated by the system comprising the holding device in accordance with an embodiment of the invention. The diameter of the artery seen in the frame shown is 4.12 mm. The entire image covers an area of about 16 mm wide by 13 mm high.

FIG. 6 depicts the velocity of the patient's blood flow on the Y-axis in cm/s and the frames representing the elapsed time on the X-axis in the sonogram depicted in FIG. 5. Velocity when combined with the diameter would allow one to calculate a volume flow rate in cubic centimeters/sec.

FIG. 7 depicts the artery diameter as a function of frames numbered 1 to 50 in the sonogram depicted in FIG. 5.

### DETAILED DESCRIPTION OF EMBODIMENTS OF THE INVENTION

The present invention provides an imaging transducer holding device for use with a patient comprising a holding structure which holds an imaging transducer and an attachment portion which attaches the holding structure to a patient.

The present invention further provides a system for determining flow mediated dilation of a blood vessel in a patient comprising a device as described above and an ultrasonic probe.

The present invention further provides a method of diagnosing flow mediated dilation of a blood vessel in a patient comprising securing a device as described above claims on the forearm of the patient, securing an ultrasonic probe into the device, and measuring the flow mediated dilation of the blood vessel.

With the holding device of the invention, the standardization and decreasing the learning curve collecting images from blood vessels were improved. CVD could be detected in the doctor's office in early stages with FMD and the holding device can help making the process smoother and/or more economical.

With the holding device of the invention, there is no need for external human manipulation during the procedure once the holding device is correctly located or positioned, thereby avoiding the error of measurement and preventing any kind if injury to the sonographist for the extended data taking cycle. The holding device fits and is attached to the patient's arm so even if the patient moves, the image is not lost, and there is no need to restart the test procedure.

The holding device of the invention improves the acquisition of images during a vascular assessment with ultrasound imaging, such as FMD, without external human manipulation, allowing for a more comfortable and reliable testing procedure. Additionally the shape of the holding device allows the sonographist to easily adjust the position and orientation of the probe before starting any measurements. The holding device is form fitting, and so it is comfortable for the patient and allows for some minor movement of the patient during the data taking process.

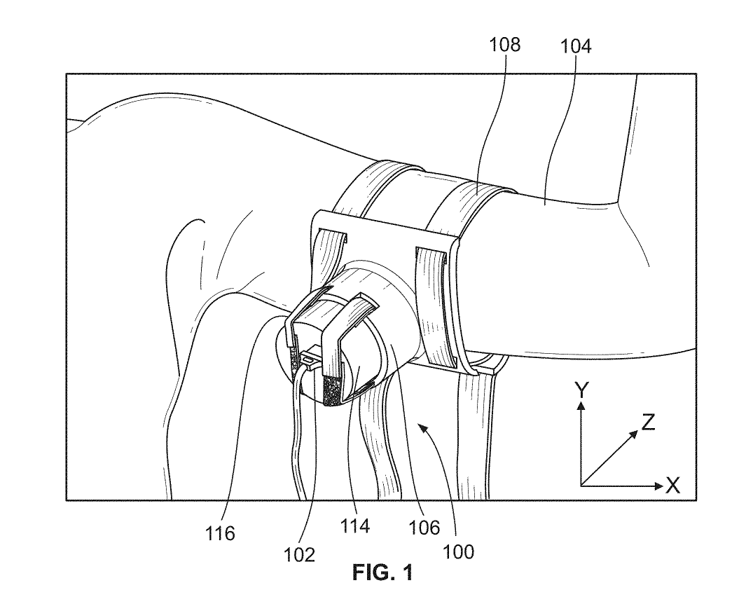

Referring to FIG. 1, one embodiment of the holding device 100 is shown. The holding device 100 may hold an imaging transducer 102. The holding device 100 may be attached to a patient to perform various procedures. In one embodiment, the holding device and imaging transducer may be used to obtain vascular information from a patient. The patient may be a human or an animal.

The holding device may be attached to the arm, leg or other part of the patient. In one embodiment, the holding device 100 is attached to the arm 104 of a patient. In one embodiment, the holding device 100 may include a holding structure 106 and an attachment portion 108. The holding structure 106 may be positioned on the inside of the patient's arm. When located on the inside of the arm, the transducer may be oriented to point toward the side and slightly to the front of the patient. In one embodiment, the attachment portion 108 may be a strap.

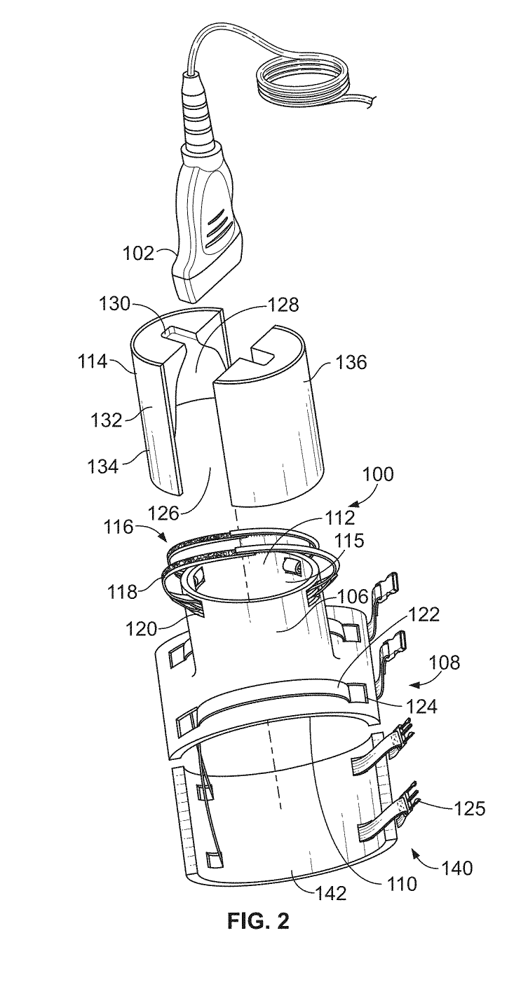

Referring to FIG. 2, a first embodiment of a holding device 100 is shown. In one embodiment, the holding device 100 may include a holding structure 106. In one embodiment, the holding structure 106 may have a surface 110. The surface 110 may correspond to the shape of the location on the patient where the holding device will be attached. For example, in one embodiment, the surface may correspond to the shape of the arm in a human. In another embodiment, the surface may correspond to the shape of a leg in a human. In one embodiment, the surface may have the dimensions of approximately 10 cm×7 cm, and a radius of curvature of approximately 50 cm. The radius of curvature at 50 cm may be larger than the radius of the average human arm or leg. However, the larger radius may provide a better and more comfortable fit because human arms and legs may not be circular in shape.

The holding structure may be made of a suitable material. In one embodiment, the holding structure may be made of plastic. Examples of plastics are noted herein. The holding structure may be made by a molding process, 3D printing, or other processes noted herein.

In one embodiment, the holding structure 106 may include an opening 112. The opening 112 may accommodate the imaging transducer 102 or the transducer holder 114. In one embodiment, the opening 112 may have an internal shape 115. In one embodiment, the internal shape 115 may correspond with the external shape of the transducer holder 114. In another embodiment, the internal shape 115 may correspond to the transducer 102. In one embodiment, the internal shape 115 may be circular. For example, the internal shape may be a cylinder and the diameter of the cylinder may be 8 cm and the height may be 6 cm. In one embodiment, the transducer holder 114 may fit snugly inside the opening 112. The user can rotate the transducer holder 114 relative to the opening 112, but the transducer holder 114 will maintain its rotational position when the user is no longer applying a rotational force.

In one embodiment, the holding structure 106 may have a securing portion 116. The securing portion 116 may be used to hold the transducer 102 or the transducer holder 114 to the holding structure 106. In one embodiment, the securing portion may be a strap 118. In one embodiment, the strap may be made of woven material or non-woven material. In one embodiment, the strap may include elastic and may stretch. The strap may include Velcro, clips, or other attachment mechanisms. In one embodiment, the strap may be tied to another strap. The securing portion may be one, two, three, or more straps. In one embodiment, the strap 118 may be connected to the holding structure 106 by one or more openings 120. In another embodiment, the securing portion 116 may be a screw or other technique to secure the transducer 102 or the transducer holder 114 to the holding structure 106.

The holding device 100 may include an attachment portion 108. The attachment portion 108 may be used to attach the holding structure 106 to the patient. In one embodiment, the attachment portion may be a strap 122. In one embodiment, the strap may be made of woven material or non-woven material. In one embodiment, the strap may include elastic and may stretch. The strap may include Velcro, clips 125, or other attachment mechanisms. In one embodiment, the strap may be tied to another strap. The attachment portion may be one, two, three, or more straps. In one embodiment, the strap 122 may be connected to the holding structure 106 by one or more openings 124.

The holding device 100 may include a transducer holder 114. The transducer holder 114 may hold the transducer 102. In one embodiment, the transducer holder 114 may have an internal cavity 126. In one embodiment, the internal cavity 126 may correspond to the shape of the transducer 102. The cavity 126 may have front opening 128. The front opening 128 may allow a portion of the transducer to extend beyond the holder 114. The cavity 126 may have a rear opening 130. The rear opening 130 may allow a portion of the transducer and/or transducer cord to extend beyond the holder 114.

The transducer holder 114 may have an external shape 132. In one embodiment, the external shape 132 corresponds with the opening 112 in the holding structure. In one embodiment, the external shape 132 may be a cylinder. In one embodiment, the transducer holder 114 may fit snugly into the opening 112.

In one embodiment, the transducer holder 114 may be two components 134, 136. In one embodiment, the two components 134, 136 may be mirror-image components. In another embodiment, the transducer holder may be a single component. The transducer holder may be made of a suitable material. In one embodiment, the transducer holder may be made of plastic. Examples of plastics are noted herein. The transducer holder may be made by a molding process, 3D printing or other processes noted herein.

The holding device 100 may allow the use of different transducers. The exterior shape of a transducer may be different for different models of the same manufacturer and the exterior shape of the transducer may be different for each manufacturer. In one embodiment, the holding device may include several different versions of the transducer holder, for example, transducer holders A, B and C. Transducer holders A, B, and C would have the same external shape 132 that would correspond to the opening 112 in the holding structure 106. However, the internal cavity would be different for holders A, B, and C, and the internal cavity would correspond to a particular transducer. For example, the internal cavity of holder A would correspond to the shape of transducer A, the internal cavity of holder B would correspond to the shape of transducer B, and the internal cavity of holder C would correspond to the shape of transducer C. The user would be able to use the same holding device and substitute the transducer holder to use a particular transducer.

The holding device 100 may include a backing portion 140. In one embodiment, the backing portion 140 may have a surface 142. The surface 142 may correspond to the shape of the location on the patient where the holding device will be attached. For example, in one embodiment, the surface may correspond to the shape of the arm in a human. In another embodiment, the surface may correspond to the shape of a leg in a human. In one embodiment, the surface may have the dimensions of approximately 10 cm×7 cm, and a radius of curvature of approximately 50 cm. The radius of curvature at 50 cm may be larger than the radius of the average human arm or leg. However, the larger radius may provide a better and more comfortable fit because human arms and legs may not be circular in shape.

The backing portion 140 may be made of a suitable material. In one embodiment, the backing portion may be made of plastic. Examples of plastics are noted herein. The backing portion may be made by a molding process, 3D printing or other processes noted herein. In other embodiments, the holding device may not include a backing portion.

The holding device 100 is able to accommodate patients with different size arms. If needed, the holding device may be made into different sizes to accommodate different sizes of patients.

The holding device 100 may be attached to the patient in the following manner. Referring to FIG. 1, the holding device 100 is positioned on the patient's body. The sonographist would move the holding device in the X and Y directions relative to the patient's body to obtain the desired position of the transducer. After achieving the desired position, the attachment portion 108 is tightened on the patient's body. The transducer holder 114 is moved in the Z direction so that the end of the transducer makes contact with the skin of the patient and the desired pressure is applied to the patient's body. The transducer holder 114 may also be rotated relative to the holding structure 106 to make angular adjustments. The proper angular orientation of a linear transducer head relative to the artery may assist in obtaining the velocity flow data or other information. After achieving the desired position, the securing portion 116 may be used to hold the transducer holder 114 in the desired position.

The holding device 100 provides a hands-free application of the transducer with X, Y, Z, and angular adjustments to alter the location and pressure applied to the patient.

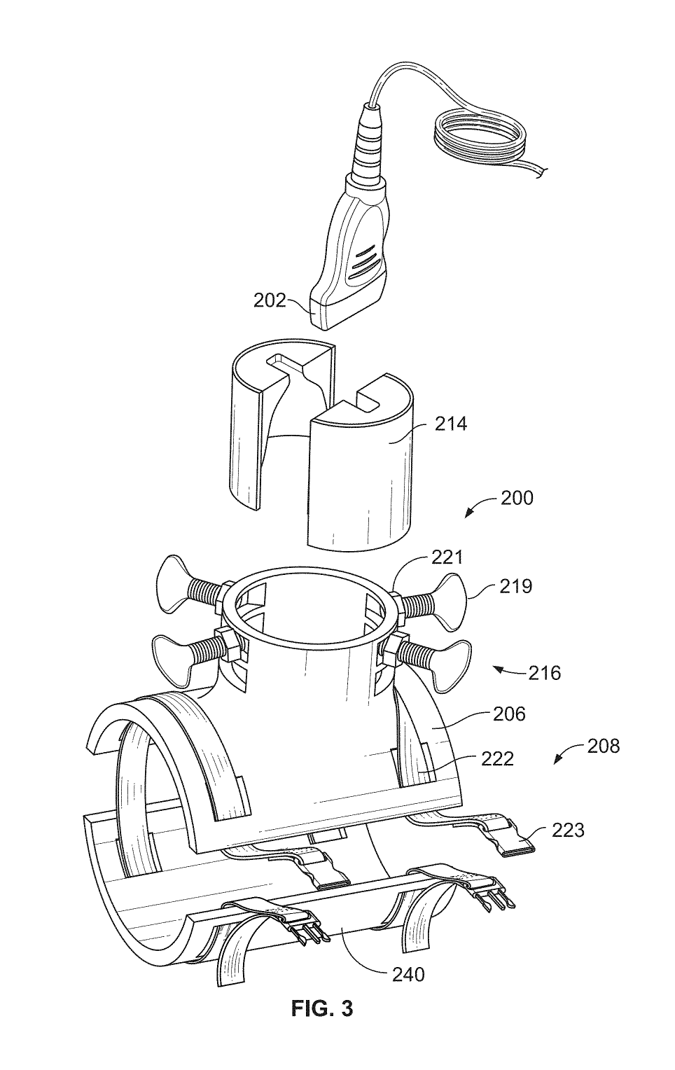

Referring to FIG. 3, a second embodiment of the holding device is shown. The holding device 200 is similar to the holding device 100 except for the securing portion 216. All of the embodiments and variations discussed with respect to FIGS. 1 and 2 are incorporated by reference with respect to FIG. 3 as appropriate.

Referring to FIG. 3, the holding device 200 may include a holding structure 206. In one embodiment, the holding structure 206 may include a securing portion 216. The securing portion 216 may be used to hold the transducer 202 or the transducer holder 214 to the holding structure 206. In one embodiment, the securing portion 216 may be a single screw 219. In other embodiments, the securing portion may include two, three, four or more screws. In one embodiment, the screw may be a thumbscrew.

The holding structure 206 may include a threaded portion 221 for the screw 219. In one embodiment, the threaded portion may be a nut 223 which is attached to the holding structure. In another embodiment, the threaded portion may be a threaded hole in the holding structure.

The holding device 200 may include an attachment portion 208. In one embodiment, the attachment portion may be a strap 222. In one embodiment, the strap 222 may include one or more clips 225.

The holding device may include a backing portion 240. In other embodiments, the holding device may not include a backing portion.

During use, the user would position the transducer holder 214 at the desired depth and angular position relative to the holding structure 206. Then the user would tighten the screw 219 to hold the transducer holder 214 in the desired position.

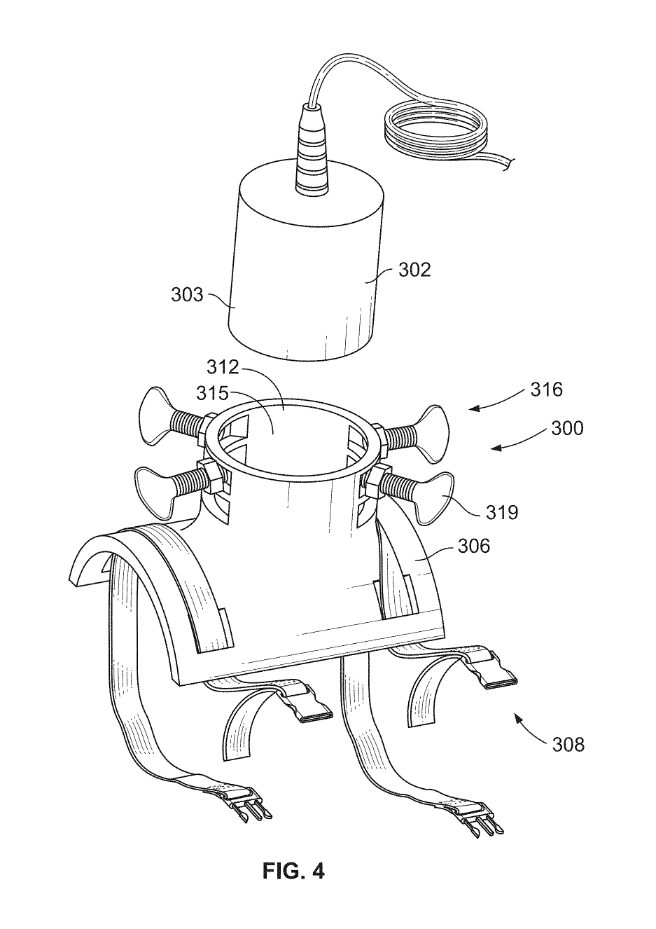

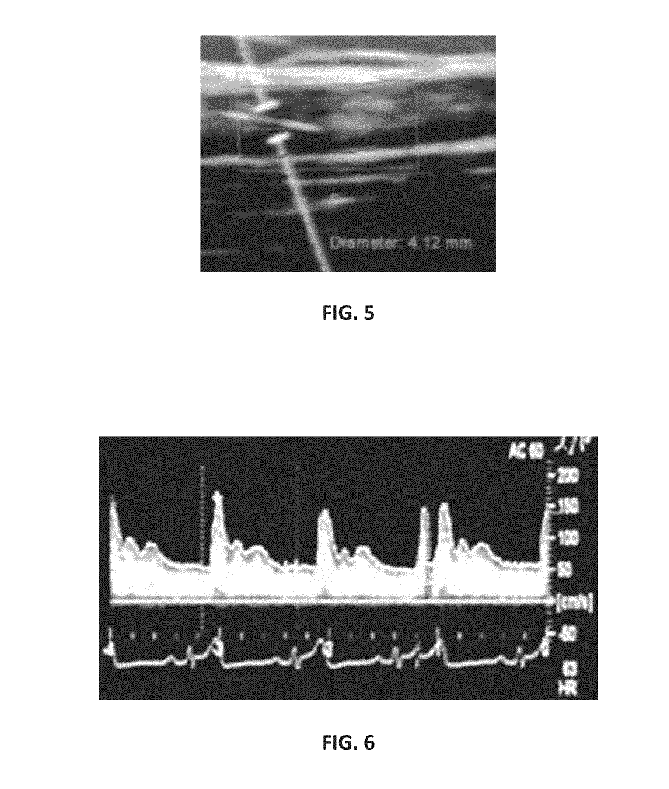

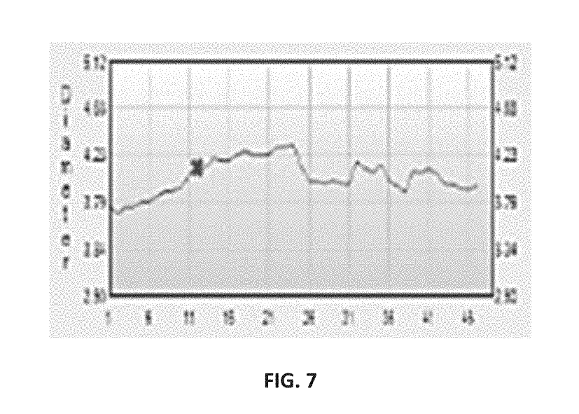

Referring to FIG. 4, a third embodiment of the holding device is shown. The holding device 300 is similar to the holding device 100 and the holding device 200 except for the shape of the imaging transducer. All of the embodiments and variations discussed with respect to FIGS. 1, 2 and 3 are incorporated by reference with respect to FIG. 4 as appropriate.

Referring to FIG. 4, the holding device 300 may include a holding structure 306. In one embodiment, the holding device 300 may include an attachment portion 308. In one embodiment, the holding structure may include a securing portion 316. In one embodiment, the securing portion may be one or more screws. In another embodiment, the securing portion may be one or more straps.

In one embodiment, the imaging transducer 302 may have an external shape 303 which corresponds to the internal shape 315 of the opening 312. In one embodiment, the transducer 302 may have a cylindrical shape and the opening 312 may have a cylindrical shape. In one embodiment, the securing portion 316, such as screw 319 may hold the transducer 302 into the desired position with respect to the holding structure 306.

In another embodiment, the external shape of the transducer may not correspond to the internal shape of the opening in the holding structure. The securing portion 316, such as one or more screws, may hold the transducer into the desired position with respect to the holding structure 306.

In one embodiment, the holding structure 306 and the imaging transducer 302 may be manufactured as a single component.

In another embodiment, the holding device 300 may include a backing portion.

In one embodiment, the imaging transducer can be a General Electric 12L Linear Array Ultrasound Probe.

The holding structure, transducer holder, and backing portion can be fabricated out of any suitable polymer, e.g., a thermoplastic or thermosetting polymer. The polymer can be petroleum based, bio-based and/or biodegradable. It is preferably made of a hypoallergenic polymer or hypoallergenic polymers.

Examples of thermoplastic polymers include polyethylene, polypropylene, polyvinyl chloride, polystyrene, polybenzimidazole, polycarbonate, bisphenol-A polysulfone, polyethersulfone, polyphenylene oxide, polyphenylene sulfide, polyether ketone, polyether ether ketone, polyimides, acrylic resins, nylons, and tetrafluoroethylene (Teflon).

Further examples of thermoplastic polymers include cellulose esters such as cellulose acetate and cellulose propionate, PLA, ABS, polyvinyl alcohol, polyethylene terephthalate, polybutylene terephthalate, as well as copolymers thereof, for example, copolymers of ethylene and propylene, ethylene and methyl methacrylate, styrene and methyl methacrylate. Polyethylene can be HDPE, MDPE, LDPE, or LLDPE. Examples of nylons include nylon 6, nylon 66, nylon 6/6-6, nylon 6/9, nylon 6/10, nylon 6/12, nylon 11, and nylon 12.

Examples of biodegradable polymers include polylactic acids, polyvinyl alcohol, and polyhydroxyalkanoates, e.g., copolymers of 3-hydroxybutyrate and 4-hydroxyvalerate. Examples of thermosetting polymers include epoxy resins, phenolic resins, unsaturated polyester resins, and polyurethanes.

The holding structure, transducer holder, and backing portion can be fabricated by any suitable technique, for example, injection molding, rotational molding, blow molding, and compression molding. In one embodiment, the holding structure, transducer holder, and/or backing portion may be fabricated by 3D printing.

The present invention further provides a system for determining flow mediated dilation of a blood vessel in a patient comprising: (i) an imaging transducer holding device comprising a holding structure which holds an imaging transducer and an attachment portion which attaches the holding structure to a patient; and (ii) an ultrasonic probe.

The system can further include a control system configured to receive and process ultrasonic echoes from the blood vessel into data indicative of flow mediated dilation of the blood vessel.

The present invention also provides a method for diagnosing the vascular condition of a patient in need thereof comprising (i) securing an imaging transducer holding device comprising a holding structure which holds an imaging transducer and an attachment portion which attaches the holding structure to a patient on the forearm of the patient; (ii) securing an ultrasonic probe into the imaging transducer holding device; and (iii) receiving and processing ultrasonic echoes from the blood vessel into data indicative of the vascular condition of the blood vessel.

In an embodiment, the vascular condition is selected from arteria carotis and arterial sclerosis. The vascular condition can be intima-media thickness (IMT) or flow mediated dilation (FMD), in particular FMD.

The following examples further illustrate the invention but, of course, should not be construed as in any way limiting its scope.

### Example 1

Flow Mediated Dilation (FMD) has immense potential to become a clinical, non-invasive biomarker of endothelial function and nitric oxide bioavailability, which regulate vasomotor activity. Unfortunately, FMD analysis techniques could deviate significantly in different laboratories if a validation process is not involved.

This example provides a validation to the assessment of FMD analysis in accordance with an embodiment of the invention and a standardization of this process before reporting results of FMD.

Brachial and femoral arteries FMD were performed on 28 apparently healthy participants (15 male and 13 female, ages 18-35 years). For the intratester reliability study, 9 subjects were asked to come to the lab for a second brachial FMD within 48 hours. All FMD procedures were performed by the same investigator, while the FMD analyses were performed by 2 independent testers who were blind to each other's analyses. FMD analyses included baseline artery diameter measurements, peak artery diameter after 5 minutes of ischemia, and FMD. Analysis was completed via an automated edge detection system by both testers after training of the methodical process of analysis to minimize variability. Intratester and intertester reliability were determined by using coefficient of variation (CV) between first and second visit (intratester) and between results obtained by both testers (intertester).

The intratester CVs for testers 1 and 2 were 3.28% and 2.62%, 3.74% and 3.27%, and 4.95% and 2.38% for the brachial baseline artery diameter, the brachial peak artery dilation, and the brachial FMD, respectively. The intertester CVs were 2.40%, 3.16%, and 3.37% for the brachial baseline artery diameter, the peak artery dilation, and the FMD, respectively and 4.52%, 5.50%, and 3.46% for the femoral baseline artery diameter, the peak artery dilation, and the FMD, respectively.

The foregoing shows that all CVs were about 5% or less, thereby confirming the strong reliability and validation of the method of the present invention. The FMD protocol using the holding device of the invention is reproducible due to the significantly low coefficient of variation. Advantageously, FMD determined using the holding device of the invention can be used as a biomarker for endothelial function.

### Example 2

This example validates the assessment of FMD analysis according to the present invention and provides a standardization for laboratories prior to reporting results of FMD.

Thirty apparently healthy individuals—15 males and 15 females, ages 18-35 years old, were recruited for this study. Exclusion criteria included known cardiovascular or cardiac disease, prescription medication medicines, and ‘over-the-counter’ painkillers, such as NSAIDs or aspirin, or nutritional supplements containing antioxidants [7]. Young female participants were tested within 4 days before or within 4 days after menses to standardize any influence that certain phases of the menstrual cycle may have on vascular physiology [18].

The investigation was approved by the Indiana State University Institutional Review Board for ethical practices and declared in accordance with the Declaration of Helinski. All subjects were tested at the same time of day to avoid any diurnal variations and following at least 8 hours of fasting [5, 11].

All participants completed a single laboratory testing session assessing baseline blood pressure (BP), heart rate (HR), and endothelium-dependent vasodilation via flow mediated dilation procedures. All laboratory testing was conducted in a darkened, quiet and temperature-controlled room approximately at 24° C. All patients were required to abstain from food and caffeine for at least 8 hours prior to testing, and a 12 hour abstinence from exercise. Ten participants were asked to return for a second visit within 48 hours after the initial assessment to repeat the procedure.

Brachial and Femoral Flow Mediated Dilation

Participants were asked to relax and lay down on an examination table. Following a 10 minute rest period, brachial blood pressure was measured in triplicate via an automated non-invasive device (Omron Automatic Blood Pressure Monitor, Omron Healthcare, Lake Forest Ill.). After assessing hemodynamic baseline conditions, a blood pressure cuff was placed on the participants' upper forearm of the right arm, just below the antecubital region of the elbow, or on the upper lower leg of the right leg, just below the patella, for brachial (b) or femoral (f) FMD, respectively. After baseline measurements were obtained, the blood pressure cuff was inflated to a supra systolic pressure (>200 mmHg) for 5 minutes, which was sufficient to provide ischemia and subsequent reactive hyperemia to the distal end of the arm and leg. FMD was performed using high resolution ultrasound (GE Logiq E, GE Medical, Milwaukee, Wis.). A 12.0 MHz linear phase array ultrasound transducer was used to image the right brachial and femoral arteries longitudinally. According to recent guidelines [5], using a 12 MHz ultrasound probe during the imaging process allows for a better definition of the endothelium at the brachial and femoral artery; however, ultrasound frequencies over 10 MHz also provide good definition, especially when using edge-detection technology. Baseline brachial and femoral artery diameter measurements were obtained utilizing an electrocardiogram (EGG) trigger system (MP150WSW, BIOPAC Systems Inc., Goleta, Calif. and Frame Grabbing and Digital Data Input modules, Medical Imaging Applications LLC, Coralville Iowa). Using an ECG-gated image selection is more time efficient as the live video stream from the imaging ultrasound feeds directly on a digital recording device using one frame per cardiac cycle. Under the present imaging method, the images were obtained using an automated imaging method, with one image being obtained once during each heart beat cycle, at the same point in the heart beat cycle. This sets the frame rate at one frame per heartbeat. The heartbeat is used to trigger the image capture, which results in consistent measurement of blood vessel diameter. Even though previous reports have selected one frame every 3 to 5 seconds [8] and even every 15 seconds [4], the use of ECG gated image selection decreases the chances to miss the peak dilation, thereby improving the accuracy.

Imaging was performed with the ultrasound probe fixed approximately 5 cm above the antecubital fossa and approximately 2 cm below the inguinal ligament for brachial and femoral measurement sites, respectively. All images were recorded continuously for 180 seconds from 30 seconds prior to cuff deflation and stored as AVI files for off-line analysis, which was performed using an automated edge-detection software (Vascular Research Tools, Medical Imaging Applications LLC, Coralville, Iowa). Brachial and femoral peak diameters were identified as the single peak diameter observed during the plateau phase after cuff deflation. All FMD procedures were completed in accordance with published guidelines [5, 11].

Analysis was performed by two independent testers who were blind to each other's analyses. Analysis included baseline artery diameter measurement, peak artery diameter post-ischemia, as well as FMD. Analysis was completed via Brachial Analyzer for Research® Software (Medical Imaging Applications LLC, Coralville, Iowa) by both testers after training of the methodical process of analysis to minimize variability (FIG. 1). After reviewing the FMD images, a square Region of Interest (ROI) was placed on the most stable region of the artery. The ROI encompassed both sides of the endothelial lumen and measured a width of at least 3 mm±0.1 mm across. The ROI was then locked, and the curvature of the ROI to endothelial lumen was checked for consistent diameter accuracy. Images with less than 75% Confidence Index of quality control were edited with computerized assistance to best fit the arterial morphology. No more than nine computerized assistances were allowed, and if more assistances were needed for obtaining accurate results, the frame was rejected. Standard deviation of the measured frame was minimized when possible, and the maximal value allowed was 0.15 mm.

FMD calculations were allometrically scaled using FMD=[Peak Diameter/(Baseline Diameter^{0.89})] proposed by Atkinson et al. [19, 20] after analyzing the slope of the relationship between logarithmic transformed baseline and peak diameters for this specific sample. In addition, FMD was presented as a percentage change from baseline measurements (% FMD) using the following formula: % FMD=[(Peak Diameter−Baseline Diameter)/(Baseline Diameter)]×100.

All variables were checked for normal distribution using Kolmogorov-Smirnov and descriptive statics, including mean and standard deviations, were obtained. A two-way ANOVA (Tester×Sex) was performed to determine differences in both brachial and femoral % FMD between testers and males and females. Intratester analysis was performed using collected data from subjects attending both brachial FMD sessions. Intertester analysis was performed using data from all 30 subjects comparing analyses between testers in both brachial and femoral assessments. Validation of the analysis technique was determined by using both intratester and intertester coefficient of variation (CV). In addition, t-tests were performed to compare intertester diameters and FMD as well as intratester CVs. All statistical analyses were performed using SPSS version 23.0 (IBM, Chicago, Ill.), and statistical significance was considered when p<0.05.

During the study, data collected during brachial FMD from one subject had some technical error on the first visit. In addition, data collected during femoral FMD from two subjects had some technical error. Data from these subjects were withdrawn from the intertester and intratester analyses. All data were normally distributed according to Kolmogorov-Smirnov analysis.

Table 1 shows the general characteristics of the sample. Males were taller, heavier, had a larger BMI, and higher systolic blood pressure than females (p<0.05).

There were no significant differences between males and females and between testers on both brachial and femoral % FMD (Table 2). In general, brachial % FMD is larger than femoral % FMD, which could be attributed to a smaller baseline diameter observed in the brachial artery.

Intratester analysis is shown on Table 3. CVs were all below 5.0% and there were no significant differences between CVs between tester 1 and tester 2 in any of the studied variables.

Intertester analysis is shown on Table 4. CVs were all at or below 5.5% and there was only one significant difference between both testers (brachial FMD).

The tests confirmed that the FMD analysis protocol of the present invention is reproducible within same tester and also that the FMD analysis protocol is reproducible between testers.

FMD protocols were carried out following international guidelines [5, 10, 11]. All images were obtained with an insonation angle of 60° and images were obtained with the help of the ECG-gated system, to avoid changes of vessel diameter observed within one cardiac cycle. These technical details provide more accurate and clear images of the studied vessel. Images were obtained by the automated imaging method described above. The recording duration of 180 seconds, 30 seconds prior to deflation accompanied by 150 seconds post-deflation, was determined to be the most adequate method in which FMD could be determined [5, 10]. The 30 seconds prior to deflation allowed for recording images of the vessel that reflect differences with baseline diameters [7, 21]. After the cuff deflation, the hyperemic blood flow rapidly increased, eliciting the vascular response, which peaked anywhere between 30 and 90 seconds in an average 18-35 years old adult. The additional 60 seconds helped to account for outliers in the vasodilatory response as well as return to baseline measurements.

The present study showed that the intratester CVs from two independent testers (tester 1 and 2) were very similar in all studied variables (range 2.62-4.95%, table 3) and there no significant difference between the CVs from both testers. Brachial baseline and peak CVs are all below 3.75%, while FMD CV was slightly higher in one of the testers (4.95%). The CVs obtained in the present study are lower than the CVs from previous reports, which are already considered as low methodological error [16, 17, 22]. Therefore, the intratester validation presented here provides data for brachial baseline and peak artery dilation, making this data an acceptable reproducible measurement of brachial artery recording. Similar results were observed for the intertester analysis (Table 4). CVs between both testers were lower or at 5.50% for all studied variables. All the studied diameters, brachial baseline and peak and femoral baseline and peak, and femoral FMD showed no significant difference between testers. Only brachial FMD showed a significant difference between testers. This difference might be the result of slightly larger peak brachial diameter observed on tester 1, which could produce a larger difference when the FMD ratio is applied. The results of the present study show that the FMD protocol used herein is reproducible within approximately 5% of variation between testers.

Table 5 shows CVs obtained using the holding device of the invention, which are lower than those in Tables 3 and 4, thereby improving the quality of the images.

This study confirmed the reproducibility of the observed results.

### LIST OF ABBREVIATIONS

BMI—Body Mass Index
BP—Blood Pressure
CV—Coefficient of Variation
ECG—Electrocardiogram
FMD—Flow Mediated Dilation
HR—Heart Rate
NO—Nitric Oxide
ROI—Region of Interest

TABLE 1
Sample demographics

| | Total | Males | Females | |
|---|---|---|---|---|
| | (n = 28) | (n = 15) | (n = 13) | P value |
| Age (yrs) | 24.01 ± 4.28 | 24.14 ± 4.88 | 23.87 ± 3.75 | 0.868 |
| Height (m) | 1.71 ± 0.09 | 1.77 ± 0.06 | 1.65 ± 0.08 | <0.001* |
| Weight (kg) | 73.36 ± 17.51 | 84.63 ± 17.2 | 62.1 ± 8.19 | <0.001* |
| BMI (kg/m²) | 25.09 ± 4.97 | 27.11 ± 5.36 | 23.08 ± 3.70 | 0.023* |
| Systolic | 109.53 ± 10.48 | 115.00 ± 9.29 | 104.07 ± 8.80 | 0.003* |
| Blood | | | | |
| Pressure | | | | |
| (mmHg) | | | | |
| Diastolic | 70.37 ± 8.53 | 70.00 ± 7.62 | 70.73 ± 9.62 | 0.819 |
| Blood | | | | |
| Pressure | | | | |
| (mmHg) | | | | |

*statistically significant between males and females

TABLE 2
Brachial and femoral percentage of flow mediated dilation in males and
females analyzed by two independent testers.

| | | | | Tester | Sex | |
|---|---|---|---|---|---|---|
| | Total | Males | Females | Effect | Effect | Interaction |
| Brachial FMD (%) | 14.9 ± 8.5 | 16.3 ± 8.8 | 33.5 ± 8.3 | 0.07 | 0.26 | 0.72 |
| Tester 1 | | | | | | |
| Brachial FMD (%) | 11.5 ± 5.2 | 12.2 ± 4.3 | 10.8 ± 6.0 | | | |
| Tester 2 | | | | | | |
| Femoral FMD (%) | 8.2 ± 8.2 | 6.9 ± 5.8 | 9.4 ± 10.0 | 0.48 | 0.34 | 0.83 |
| Tester 1 | | | | | | |
| Femoral FMD (%) | 9.7 ± 7.5 | 8.9 ± 4.2 | 10.5 ± 9.8 | | | |
| Tester 2 | | | | | | |

TABLE 3
Intratester analysis on both testers (n = 9)

| | Tester 1 | Tester 2 | |
|---|---|---|---|
| | (% CV) | (% CV) | P |
| Brachial baseline | 3.28 ± 3.63 | 2.62 ± 2.61 | 0.68 |
| Brachial peak | 3.74 ± 4.90 | 3.27 ± 2.01 | 0.75 |
| Brachial FMD | 4.95 ± 5.83 | 2.38 ± 1.79 | 0.26 |

% CV: coefficient of variation in percentage

TABLE 4
Intertester analysis (n = 28)

| | | | | (% CV between |
|---|---|---|---|---|
| | Tester 1 | Tester 2 | P | T1 and T2) |
| Brachial | 3.62 ± 0.56 | 3.60 ± 0.59 | 0.89 | 2.40% |
| baseline (mm) | | | | |
| Brachial peak | 4.14 ± 0.51 | 4.00 ± 0.59 | 0.37 | 3.16% |
| (mm) | | | | |
| Brachial FMD | 1.32 ± 0.86 | 1.28 ± 0.55* | 0.04 | 3.25% |
| (ratio) | | | | |
| Femoral | 5.95 ± 1.16 | 6.20 ± 0.91 | 0.39 | 4.52% |
| baseline (mm) | | | | |
| Femoral peak | 6.41 ± 1.16 | 6.78 ± 0.89 | 0.20 | 5.50% |
| (mm) | | | | |
| Femoral FMD | 1.31 ± 0.95 | 1.34 ± 0.83 | 0.30 | 3.61% |
| (ratio) | | | | |

*statistically significant difference between tester 1 and tester 2.

TABLE 5

| | Subject* | | | | | | | | | | |
|---|---|---|---|---|---|---|---|---|---|---|---|
| | Male/ | | Basal | Basal | CV | Peak 1 | Peak 2 | CV | FMD 1 | FMD 2 | CV |
| | Female | Age | 1 (mr) | 2 (mr) | Basal | (mn) | (mn) | Peak | (%) | (%) | FMD |
| 1 | 1 | 19 | 3.18 | 3.36 | 3.9% | 3.58 | 3.74 | 3.1% | 1.125786 | 1.113095 | 0.8% |
| 2 | 0 | 20 | 4.74 | 4.46 | 4.3% | 4.99 | 4.83 | 2.3% | 1.052743 | 1.08296 | 2.0% |
| 3 | 0 | 19 | 4.19 | 4.25 | 1.0% | 4.29 | 4.41 | 2.0% | 1.023866 | 1.037647 | 0.9% |
| | | | | | 3.1% | | | 2.4% | | | 1.2% |
| | | | | | (avg) | | | (avg) | | | (avg) |

*1 = Male, 0 = Female

### REFERENCES

1. Flammer A J, Anderson T, Celermajer D S, Creager M A, Deanfield J, Ganz P, Hamburg N M, Luscher I F, Shechter M, Taddei S et al: The assessment of endothelial function: from research into clinical practice. Circulation 2012, 126(6):753-767.
2. Rosamond W, Flegal K, Friday G, Furie K, Go A, Greenland K, Haase N, Ho M, Howard V, Kissela B., et al: Heart disease and stroke statistics—2007 update: a report from the American Heart Association Statistics Committee and Stroke Statistics Subcommittee. Circulation 2007, 115(5):e69-171.
3. Libby P, Ridker P M, Maseri A: Inflammation and atherosclerosis. Circulation 2002, 105(9):1135-1143.
4. Celermajer D S, Sorensen K E, Gooch V M, Spiegelhalter D J, Miller 01, Sullivan I D, Lloyd J K, Deanfield J E: Non-invasive detection of endothelial dysfunction in children and adults at risk of atherosclerosis. Lancet 1992, 340(8828):1111-1115.
5. Harris R A, Nishiyama S K, Wray D W, Richardson R S: Ultrasound Assessment of Flow-Mediated Dilation. Hypertension 2010, 55(5):1075-1085.
6. Corretti M C, Anderson T J, Benjamin E J, Celermajer D, Charbonneau F, Creager M A, Deanfield J, Drexler H, Gerhard-Herman M, Herrington D et al: Guidelines for the ultrasound assessment of endothelial-dependent flow-mediated vasodilation of the brachial artery: a report of the International Brachial Artery Reactivity Task Force. Journal of the American College of Cardiology 2002, 39(2):257-265.
7. Verma S, Buchanan M R, Anderson T J: Endothelial function testing as a biomarker of vascular disease. Circulation 2003, 108(17):2054-2059.
8. Gurovich A N, Braith R W: Enhanced external counterpulsation creates acute blood flow patterns responsible for improved flow-mediated dilation in humans. Hypertension research: official journal of the Japanese Society of Hypertension 2013, 36(4):297-305.
9. Morishima T, Restaino R M, Walsh L K, Kanaley J A, Padilla J: Prior exercise and standing as strategies to circumvent sitting-induced leg endothelial dysfunction. Clin Sci (Land) 2017, 131(11): 1045-1053.
10. Rodriguez-Miguelez P, Seigler N, Harris R A: Ultrasound Assessment of Endothelial Function: A Technical Guideline of the Flow-mediated Dilation Test. J Vis Exp 2016(110).
11. Thijssen D H, Black M A, Pyke K E, Padilla J, Atkinson G, Harris R A, Parker B, Widlansky M E, Tschakovsky M E, Green D J: Assessment of flow-mediated dilation in humans: a methodological and physiological guideline. Am J Physiol Heart Circ Physiol 2011, 300(1):H2-12.
12. Ghiadoni L, Salvetti M, Muiesan M L, Taddei S: Evaluation of endothelial function by flow mediated dilation: methodological issues and clinical importance. High Blood Press Cardiovasc Prev 2015, 22(1):17-22.
13. Gori T, Dragoni S, Lisi M, Di Stolfo G, Sonnati S, Fineschi M, Parker J D: Conduit artery constriction mediated by low flow a novel noninvasive method for the assessment of vascular function. Journal of the American College of Cardiology 2008, 51(20):1953-1958.
14. Gurovich A N, Avery J C, Holtgrieve N B, Braith R W: Flow-mediated dilation is associated with endothelial oxidative stress in human venous endothelial cells. Vase Med 2014,19(4):251-256.
15. Sonka M, Liang W, Lauer R M: Automated analysis of brachial ultrasound image sequences: early detection of cardiovascular disease via surrogates of endothelial function. IEEE transactions on medical imaging 2002, 21(10):1271-1279.
16. Avery J C, Gurovich A N, Braith R W: Reproducibility Of Brachial And Femoral Arterial Diameter And Flow-mediated Dilation Via Automatic Edge-detection Software. Medicine & Science in Sports & Exercise 2010, 42(5):315.
17. Woodman R, Playford D, Watts G, Cheetham C, Reed C, Taylor R, Puddey I, Beilin L, Burke V, Mori I: Improved analysis of brachial artery ultrasound using a novel edge-detection software system. Journal of Applied Physiology 2001, 91(2):929-937.
18. Adkisson E J, Casey D. P., Beck D. T., Gurovich A. N., Martin J. S., Braith R. W.: Central, Peripheral, and Resistance Arterial Reactivity Fluctuates During The Phases of The Menstrual Cycle. Experimental Biology and Medicine 2010, 235:111-118.
19. Atkinson G, Batterham A M: The percentage flow-mediated dilation index: a large-sample investigation of its appropriateness, potential for bias and causal nexus in vascular medicine. Vase Med 2013, 18(6):354-365.
20. Atkinson G, Batterham A M, Thijssen D H, Green D J: A new approach to improve the specificity of flow-mediated dilation for indicating endothelial function in cardiovascular research. J
21. Hypertens 2013, 31(2):287-291.
22. Dawson E A, Alkarmi A, Thijssen D H, Rathore S, Marsman D E, Cable N T, Wright D J, Green D J:
23. Low-flow mediated constriction is endothelium-dependent: effects of exercise training after radial artery catheterization. Circ Cardiovasc Intery 2012, 5(5):713-719.
24. Ghiadoni L, Faita F, Salvetti M, Cordiano C, Biggi A, Puato M, Di Monaco A, De Siati L, Volpe M, Ambrosio G et al: Assessment of flow-mediated dilation reproducibility: a nationwide multicenter study. Journal of hypertension 2012, 30(7):1399-1405.
25. Al Mheid I, Quyyumi A A: Allometric scaling of endothelium-dependent vasodilation: brachial artery flow-mediated dilation coming of age? Vase Med 2013, 18(6):368-371.
26. Atkinson G: The dependence of FMD % on baseline diameter: a problem solved by allometric scaling. Clin Sci (Land) 2013, 125(1):53-54.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An imaging transducer holding device for use with a patient, comprising:
a holding structure which holds an imaging transducer;
an attachment portion which attaches the holding structure to a patient.

2. The holding device according to claim 1, wherein the holding structure includes an opening for the imaging transducer.

3. The holding device according to claim 1, wherein the holding structure is shaped to fit the patient.

4. The holding device according to claim 1, wherein the attachment portion is a strap.

5. The holding device according to claim 1, wherein the holding structure includes a securing portion to secure an imaging transducer in the holding structure.

6. The holding device according to claim 5, wherein the securing portion is a strap.

7. The holding device according to claim 5, wherein the securing portion is screw.

8. The holding device according to claim 1, further comprising a transducer holder.

9. The holding device according to claim 8, wherein the transducer holder moves relative to the holding structure.

10. The holding device according to claim 8, wherein the transducer holder moves linearly relative to the holding structure.

11. The holding device according to claim 8, wherein the transducer holder moves rotationally relative to the holding structure.

12. The holding device according to claim 1 further comprising a backing portion.

13. The holding device according to claim 1, wherein the holding structure portion is made by injection molding, blow molding, rotational molding or compression molding or by 3D printing.

14. The holding device according to claim 1, wherein the holding structure is made of one or more plastic materials.

15. A system for determining flow mediated dilation of a blood vessel in a patient comprising:
(i) an imaging transducer holding device comprising a holding structure which holds an imaging transducer and an attachment portion which attaches the holding structure to a patient;
and
(ii) an ultrasonic probe.

16. The system according to claim 15, further including a control system configured to receive and process ultrasonic echoes from the blood vessel into data indicative of flow mediated dilation of the blood vessel.

17. A method for diagnosing the vascular condition of a patient in need thereof comprising:
(i) securing an imaging transducer holding device comprising a holding structure which holds an imaging transducer and an attachment portion which attaches the holding structure to a patient on the forearm of the patient;
(ii) securing an ultrasonic probe into the imaging transducer holding device; and
(iii) receiving and processing ultrasonic echoes from the blood vessel into data indicative of the vascular condition of the blood vessel.

18. The method according to claim 17, wherein the vascular condition is selected from arteria carotis and arterial sclerosis.

19. The method according to claim 17, wherein the vascular condition is intima-media thickness (IMT) or flow mediated dilation (FMD).

20. The method according to claim 19, wherein the vascular condition is flow mediated dilation (FMD).
